(19) United States
(12) Patent Application Publication
(10) Pub. No.: US 2026/0053512 A1
(43) Pub. Date: Feb. 26, 2026

(54) JIGS AND RELATED METHODS FOR GUIDING A CUTTING INSTRUMENT FOR CONTROLLED SURGICAL CUTTING OF A BODY PORTION OF A PATIENT

(71) Applicant: Ohio State Innovation Foundation, Columbus, OH (US)

(72) Inventor: Said ATWAY, Columbus, OH (US)

(21) Appl. No.: 19/102,613

(22) PCT Filed: Aug. 8, 2023
(86) PCT No.: PCT/US2023/029744

§ 371 Date: Feb. 10, 2025

Related U.S. Application Data
(60) Provisional application No. 63/370,711, filed on Aug. 8, 2022.

(51) Int. Cl. A61B 17/17; A61B 17/00; A61B 17/16; A61B 17/56; A61B 90/00; A61B 90/50

(52) U.S. Cl. CPC A61B 17/1775; A61B 17/1613; A61B 17/56; A61B 90/08; A61B 90/50; A61B 2017/00477; A61B 2017/565; A61B 2090/0807

(57) ABSTRACT

A jig for guiding a cutting instrument for controlled surgical cutting of a body portion of a patient may include an adjustable support structure configured for positioning relative to the patient, an angulation assembly coupled to the support structure, and a guide coupled to the support structure and spaced apart from the angulation assembly. The cutting instrument may include a driver and a cutting burr. The angulation assembly may be configured for receiving a distal portion of the driver therein and controlling angular movement of the cutting instrument about a central point defined by the angulation assembly in a plurality of planes orthogonal to one another. The guide may define an opening configured for receiving a proximal portion of the driver therein and controlling movement of the proximal portion of the driver along a first predefined path such that the cutting burr moves along a second predefined path.

### CROSS REFERENCE TO RELATED APPLICATIONS

This application claims priority to and benefit of U.S. Provisional Application No. 63/370,711, filed Aug. 8, 2022, which is incorporated herein by reference in its entirety.

### FIELD

The present disclosure relates generally to medical devices and more particularly to jigs and related methods for guiding a cutting instrument for controlled surgical cutting of a body portion of a patient.

### BACKGROUND

Various types of surgery may involve cutting a body portion of a patient for corrective purposes. For instance, certain surgical procedures may include performing an osteotomy, which is a bone-cutting procedure for realigning and/or reshaping one or more bones and/or joints of a patient. Common surgical sites for an osteotomy include the toes, feet, legs, knees, hips shoulders, elbows, jaw, and spine of a patient. As a particular example, an osteotomy often may be performed on a patient's toe for correction of a bunion, also known as hallux valgus, which may form due to a deviated position of the big toe toward the second toe.

In recent years, bunion surgery has focused on three-dimensional correction. According to one longstanding approach, referred to as the Lapidus fusion procedure, bunion correction may be achieved by removing the cartilage surfaces from the metatarsal and the medial cuneiform, correcting the angular deformity, and then placing hardware to facilitate fusion between the bones at the tarsometatarsal joint. Although the Lapidus procedure is not new, the concept of utilizing a cutting guide and specialized instrumentation to facilitate this joint-sacrifice technique has led to increased popularity among surgeons in view of increased precision and efficiency of the procedure. However, the fusion resulting from the Lapidus procedure can lead to instability of the surrounding joints and increased risk of other issues.

According to an alternative procedure, bunion correction may be achieved in a minimally invasive manner by performing an osteotomy of the metatarsal and then realigning and fixating the cut bone segments with hardware. Notably, this minimally invasive approach preserves the patient's joint, but it does not provide controlled three-dimensional correction. A particular challenge relates to precision of the osteotomy, as the surgeon typically uses a cutting instrument in a freehand manner to cut the bone, requiring significant experience and a high degree of skill to make a desired cut. In some instances, the surgeon may seek to form a crescentic cut, as this has been found to be one of the most effective cut shapes for recreating the metatarsal and preserving the joint. However, a crescentic cut can be one of the most difficult types of cuts, particularly when using a minimally invasive approach, and thus may be time consuming to achieve in a precise manner. In other instances, the surgeon may cut the metatarsal in different shapes that are easier to form but may complicate realignment and/or fixation of the cut bone segments. In view of the steep learning curve in forming a precise crescentic cut and the tradeoffs associated with other shapes of cuts, many surgeons may be deterred from performing bunion correction in a minimally invasive fashion. Despite continued popularity of minimally invasive surgery, existing techniques lack a means for precisely forming a desired cut shape, such as a crescentic cut, using a minimally invasive approach. Moreover, current techniques lack a means for objectively determining the depth and angle of a cut made in a minimally invasive fashion, often requiring the surgeon to rely on feel and experience. Notably, the above-mentioned challenges are not limited to performing an osteotomy for bunion correction but rather are similarly applicable to osteotomies performed on other bones as well as cutting of other body portions of a patient in accordance with various procedures for which precise and efficient cutting is desirable.

A need therefore exists for improved devices and methods for guiding a cutting instrument for controlled surgical cutting of a body portion of a patient, such as when performing an osteotomy.

### SUMMARY

The present disclosure provides jigs and related methods for guiding a cutting instrument for controlled surgical cutting of a body portion of a patient.

In one aspect, a jig for guiding a cutting instrument for controlled surgical cutting of a body portion of a patient is provided, with the cutting instrument including a driver and a cutting burr coupled to the driver. In some implementations, the jig may include an adjustable support structure configured for positioning relative to the patient, an angulation assembly coupled to the adjustable support structure, and a guide coupled to the adjustable support structure and spaced apart from the angulation assembly. The angulation assembly may be configured for receiving a distal portion of the driver therein and controlling angular movement of the cutting instrument about a central point defined by the angulation assembly in a plurality of planes orthogonal to one another. The guide may define an opening extending therethrough, and the opening may be configured for receiving a proximal portion of the driver therein and controlling movement of the proximal portion of the driver along a first predefined path such that the cutting burr moves along a second predefined path.

In some implementations, the adjustable support structure may include a plurality of supports, and one or more coupling assemblies each adjustably coupling two of the supports to one another. In some implementations, the plurality of supports may include a pair of first supports extending parallel to one another, and a second support coupled to each of the first supports and extending transverse to each of the first supports. In some implementations, the first supports each may include a plurality of first markings, and the second support may include a plurality of second markings. In some implementations, the first markings may correspond to an angle of approach of the cutting instrument toward the body portion of the patient or a cutting depth of the cutting instrument into the body portion of the patient, and the second markings may correspond to the angle of approach of the cutting instrument toward the body portion of the patient or the cutting depth of the cutting instrument into the body portion of the patient.

In some implementations, the one or more coupling assemblies may include a first coupling assembly adjustably coupling the second support to one of the first supports, and a second coupling assembly adjustably coupling the second support to the other of the first supports. In some implementations, the first coupling assembly may be configured for allowing the second support to translate relative to the one of the first supports, and the second coupling assembly may be configured for allowing the second support to translate relative to the other of the first supports. In some implementations, the first coupling assembly may be configured for allowing the second support to rotate relative to the one of the first supports, and the second coupling assembly may be configured for allowing the second support to rotate relative to the other of the first supports. In some implementations, the adjustable support structure also may include a first locking assembly and a second locking assembly. The first locking assembly may be configured for moving between a locked configuration, in which the first locking assembly prevents adjustment of the second support and the one of the first supports relative to one another, and an unlocked configuration, in which the first locking assembly permits adjustment of the second support and the one of the first supports relative to one another via the first coupling assembly. The second locking assembly may be configured for moving between a locked configuration, in which the second locking assembly prevents adjustment of the second support and the other of the first supports relative to one another, and an unlocked configuration, in which the second locking assembly permits adjustment of the second support and the other of the first supports relative to one another via the second coupling assembly.

In some implementations, the adjustable support structure may be configured for removably attaching to a bed configured for receiving the patient thereon. In some implementations, the adjustable support structure may be configured for removably attaching to a table configured for receiving the patient thereon. In some implementations, the adjustable support structure may be configured for removably attaching to the patient. In some implementations, the jig also may include a base, and the adjustable support structure may be fixedly attached to the base. In some implementations, the base may include a baseplate having a planar shape, and the adjustable support structure may be fixedly attached to the baseplate by one or more mounts. In some implementations, the adjustable support structure may be configured for adjusting an angle of approach of the cutting instrument toward the body portion of the patient. In some implementations, the adjustable support structure may be configured for adjusting a cutting depth of the cutting instrument into the body portion of the patient.

In some implementations, the angulation assembly may be fixedly coupled to the adjustable support structure. In some implementations, the angulation assembly may be movably coupled to the adjustable support structure. In some implementations, the angulation assembly may be movably coupled to a support of the adjustable support structure. In some implementations, the angulation assembly may be configured for translating relative to the support of the adjustable support structure. In some implementations, the adjustable support structure also may include one or more clamps configured for moving between a locked configuration, in which the one or more clamps prevents movement of the angulation assembly relative to the adjustable support structure, and an unlocked configuration, in which the one or more clamps permits movement of the angulation assembly relative to the adjustable support structure.

In some implementations, the angulation assembly may include a bearing configured for receiving the distal portion of the driver therein and controlling angular movement of the cutting instrument about the central point in the plurality of planes orthogonal to one another. In some implementations, the central point may be defined by the bearing. In some implementations, the bearing may be a spherical bearing comprising an inner ring having a partial-spherical outer surface, and an outer ring having a partial-spherical inner surface. In some implementations, the angulation assembly also may include a housing, and the bearing may be disposed at least partially within the housing. In some implementations, the angulation assembly also may include a mounting ring coupled to the adjustable support structure. In some implementations, the mounting ring may encircle a support of the adjustable support structure. In some implementations, the mounting ring may be configured for translating along the support of the adjustable support structure.

In some implementations, the guide may be fixedly coupled to the adjustable support frame. In some implementations, the guide may be movably coupled to the adjustable support frame. In some implementations, the guide may be movably coupled to a support of the adjustable support frame. In some implementations, the guide may be configured for translating relative to the support of the adjustable support frame. In some implementations, the adjustable support structure also may include one or more clamps configured for moving between a locked configuration, in which the one or more clamps prevents movement of the guide relative to the adjustable support structure, and an unlocked configuration, in which the one or more clamps permits movement of the guide relative to the adjustable support structure.

In some implementations, the guide may include a guide plate, and the guide plate may define the opening. In some implementations, the guide plate may include a first surface and a second surface disposed opposite one another, and the opening may extend from the first surface to the second surface. In some implementations, the first surface may face away from the angulation assembly, and the second surface may face toward the angulation assembly. In some implementations, the guide plate may have a curved shape such that each of the first surface and the second surface is a curved surface. In some implementations, the first surface may be a convex surface, and wherein the second surface may be a concave surface. In some implementations, the guide also may include a mounting ring coupled to the adjustable support structure. In some implementations, the mounting ring may encircle a support of the adjustable support structure. In some implementations, the mounting ring may be configured for translating along the support of the adjustable support structure.

In some implementations, the opening may have an elongate shape such that each of the first predefined path and the second predefined path has an elongate shape. In some implementations, the opening may have a linear shape such that each of the first predefined path and the second predefined path has a linear shape. In some implementations, the opening may have a contoured shape such that each of the first predefined path and the second predefined path has a contoured shape. In some implementations, the opening may have a curved shape such that each of the first predefined path and the second predefined path has a curved shape. In some implementations, the opening may have a crescentic shape such that each of the first predefined path and the second predefined path has a crescentic shape. In some implementations, the first predefined path and the second predefined path may have the same shape. In some implementations, the first predefined path may have a first size, and the second predefined path may have a second size that is different than the first size. In some implementations, the second size may be less than the first size. In some implementations, the second predefined path may be inverted relative to the first predefined path.

In another aspect, a system for controlled surgical cutting of a body portion of a patient is provided. In some implementations, the system may include a cutting instrument and a jig configured for mounting the cutting instrument thereto. The cutting instrument may include a driver and a cutting burr coupled to the driver. The jig may include an adjustable support structure configured for positioning relative to the patient, an angulation assembly coupled to the adjustable support structure, and a guide coupled to the adjustable support structure and spaced apart from the angulation assembly. The angulation assembly may be configured for receiving a distal portion of the driver therein and controlling angular movement of the cutting instrument about a central point defined by the angulation assembly in a plurality of planes orthogonal to one another. The guide may define an opening extending therethrough, and the opening may be configured for receiving a proximal portion of the driver therein and controlling movement of the proximal portion of the driver along a first predefined path such that the cutting burr moves along a second predefined path.

In some implementations, the adjustable support structure may include a plurality of supports, one or more coupling assemblies each adjustably coupling two of the supports to one another, and one of more locking assemblies each configured for moving between a locked configuration, in which the locking assembly prevents adjustment of two of the supports relative to one another, and an unlocked configuration, in which the locking assembly permits adjustment of the two of the supports relative to one another via the one or more coupling assemblies. In some implementations, the angulation assembly may include a bearing configured for receiving the distal portion of the driver therein and controlling angular movement of the cutting instrument about the central point in the plurality of planes orthogonal to one another. In some implementations, the bearing may be a spherical bearing including an inner ring having a partial-spherical outer surface, and an outer ring having a partial-spherical inner surface.

In some implementations, the opening may have an elongate shape such that each of the first predefined path and the second predefined path has an elongate shape. In some implementations, the opening may have a linear shape such that each of the first predefined path and the second predefined path has a linear shape. In some implementations, the opening may have a contoured shape such that each of the first predefined path and the second predefined path has a contoured shape. In some implementations, the opening may have a curved shape such that each of the first predefined path and the second predefined path has a curved shape. In some implementations, the opening may have a crescentic shape such that each of the first predefined path and the second predefined path has a crescentic shape. In some implementations, the first predefined path and the second predefined path may have the same shape, and the second predefined path may be inverted relative to the first predefined path.

In yet another aspect, a method for guiding a cutting instrument for controlled surgical cutting of a body portion of a patient is provided, with the cutting instrument including a driver and a cutting burr coupled to the driver. The method may include mounting the cutting instrument to a jig. The jig may include an adjustable support structure configured for positioning relative to the patient, an angulation assembly coupled to the adjustable support structure, and a guide coupled to the adjustable support structure and spaced apart from the angulation assembly. The angulation assembly may receive a distal portion of the driver therein and control angular movement of the cutting instrument about a central point defined by the angulation assembly in a plurality of planes orthogonal to one another. The guide may define an opening extending therethrough, and the opening may receive a proximal portion of the driver therein and control movement of the proximal portion of the driver along a first predefined path such that the cutting burr moves along a second predefined path.

In some implementations, the body portion of the patient may be a bone of the patient. In some implementations, the body portion of the patient may be a bone of the patient's toes, feet, legs, knees, hips, shoulders, jaw, or spine. In some implementations, the surgical cutting may be an osteotomy. In some implementations, the opening may have an elongate shape such that each of the first predefined path and the second predefined path has an elongate shape. In some implementations, the opening may have a linear shape such that each of the first predefined path and the second predefined path has a linear shape. In some implementations, the opening may have a contoured shape such that each of the first predefined path and the second predefined path has a contoured shape. In some implementations, the opening may have a curved shape such that each of the first predefined path and the second predefined path has a curved shape. In some implementations, the opening may have a crescentic shape such that each of the first predefined path and the second predefined path has a crescentic shape. In some implementations, the first predefined path and the second predefined path may have the same shape, and the second predefined path may be inverted relative to the first predefined path. In some implementations, the surgical cutting may include performing a metatarsal osteotomy of the patient's foot. In some implementations, the surgical cutting may include performing a lesser metatarsal osteotomy of the patient's foot. In some implementations, the surgical cutting may include performing a fifth metatarsal osteotomy of the patient's foot. In some implementations, the surgical cutting may include performing a calcaneal osteotomy of the patient's foot. In some implementations, the surgical cutting may include performing a phalangeal osteotomy of the patient's foot. In some implementations, the surgical cutting may include performing a wedge resection and alignment procedure for fusion of the patient's foot and ankle.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a top view of a metatarsal having a bunion, showing an example location of a cut to be made for performing an osteotomy for bunion correction.

FIG. 1B is a side view of the metatarsal of FIG. 1A, showing the location of the cut to be made for performing the osteotomy.

FIG. 1C is a top view of the metatarsal of FIG. 1A after performing the osteotomy, realigning the cut bone segments, and fixating the cut bone segments with a bone screw

FIG. 1D is a side view of the metatarsal of FIG. 1A after performing the osteotomy, realigning the cut bone segments, and fixating the cut bone segments with the bone screw.

FIG. 2A is a perspective view of an example jig for guiding a cutting instrument for controlled surgical cutting of a body portion of a patient in accordance with implementations of the disclosure, showing an example cutting instrument mounted to the jig and a model of a portion of a human foot positioned relative to the jig for performing an osteotomy for bunion correction.

FIG. 2B is a perspective view of the jig of FIG. 2A, the cutting instrument, and the model of the portion of the human foot.

FIG. 2C is a side view of the jig of FIG. 2A, the cutting instrument, and the model of the portion of the human foot.

FIG. 2D is a side view of the jig of FIG. 2A, the cutting instrument, and the model of the portion of the human foot.

FIG. 2E is a front view of the jig of FIG. 2A, the cutting instrument, and the model of the portion of the human foot.

FIG. 2F is a rear view of the jig of FIG. 2A, the cutting instrument, and the model of the portion of the human foot.

FIG. 2G is a top view of the jig of FIG. 2A, the cutting instrument, and the model of the portion of the human foot.

The detailed description is set forth with reference to the accompanying drawings. The drawings are provided for purposes of illustration only and merely depict example implementations of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various implementations may utilize elements or components other than those illustrated in the drawings, and some elements and/or components may not be present in various implementations. The use of singular terminology to describe a component or element may, depending on the context, encompass a plural number of such components or elements and vice versa.

### DETAILED DESCRIPTION

In the following description, specific details are set forth describing some implementations consistent with the present disclosure. Numerous specific details are set forth in order to provide a thorough understanding of the implementations. It will be apparent, however, to one skilled in the art that some implementations may be practiced without some or all of these specific details. The specific implementations disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one implementation may be incorporated into other implementations unless specifically described otherwise or if the one or more features would make an implementation non-functional. In some instances, well known methods, procedures, components, and circuits have not been described in detail so as not to unnecessarily obscure aspects of the implementations.

### Overview

The present disclosure provides jigs and related methods for guiding a cutting instrument for controlled surgical cutting of a body portion of a patient. The jigs and methods described herein may allow for a three-dimensional corrective procedure using a controlled cutting guide. In some implementations, the disclosed jigs and methods may be used for performing a minimally invasive, three-dimensional osteotomy, such as for bunion correction. The disclosed technology may be considered disruptive to the above-described, current standard of using a less-precise osteotomy for correcting a bunion through a minimally invasive approach. In other implementations, the disclosed jigs and methods may be used for other types of surgical cutting of various types of body parts, particularly in instances in which controlled, precise cutting is desirable Ultimately, the disclosed jigs and methods may provide both surgeons and patients a secure, predictable and safe technology that can be relied upon for reproducible results.

As described below, the disclosed technology may allow for an external guide to correct a deformity, such as a foot deformity, limiting the need for large incisions while still providing a precise guide for surgeons. By using a cutting guide that would function outside of the skin the surgeon will have control in reducing the deformity in multiple planes while utilizing a less invasive approach. In some implementations, the cutting guide may be used to make a cut in a crescentic fashion, which may allow surgeons to correct the deformity in all three body planes, without sacrificing a joint. As described herein, the disclosed osteotomy guide may provide a reproducible way of correcting bunions without a large invasive fusion.

In some instances, the disclosed jigs may be described herein as being used for performing an osteotomy on a metatarsal for bunion correction. It will be appreciated, however, that the scope of the present disclosure is not so limited. Rather, the jigs described herein may be adapted and used for guiding a cutting instrument for controlled cutting of various body portions of a patient in accordance with different types of surgical procedures.

### Example Jigs and Methods

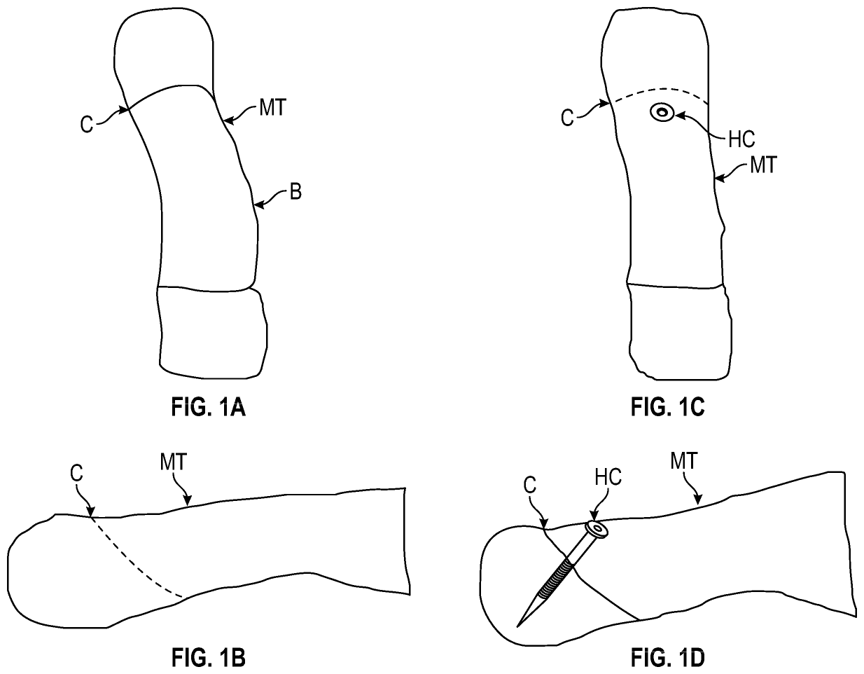

FIGS. 1A-1D schematically illustrate an example of bunion correction in accordance with implementations of the disclosure. As described below, in some instances, the disclosed jigs and methods may be used for performing an osteotomy to facilitate bunion correction, although various other uses are envisioned. FIGS. 1A and 1B illustrate a metatarsal MT having a bunion B, showing an example location of a cut C to be made for performing an osteotomy for bunion correction. FIGS. 1C and 1D illustrate the metatarsal MT following correction of the bunion B. Surgical correction, which may be performed in a minimally invasive manner, generally may include performing the osteotomy, realigning the cut bone segments of the metatarsal MT, and fixating the cut bone segments with one or more hardware components HC, such as a bone screw.

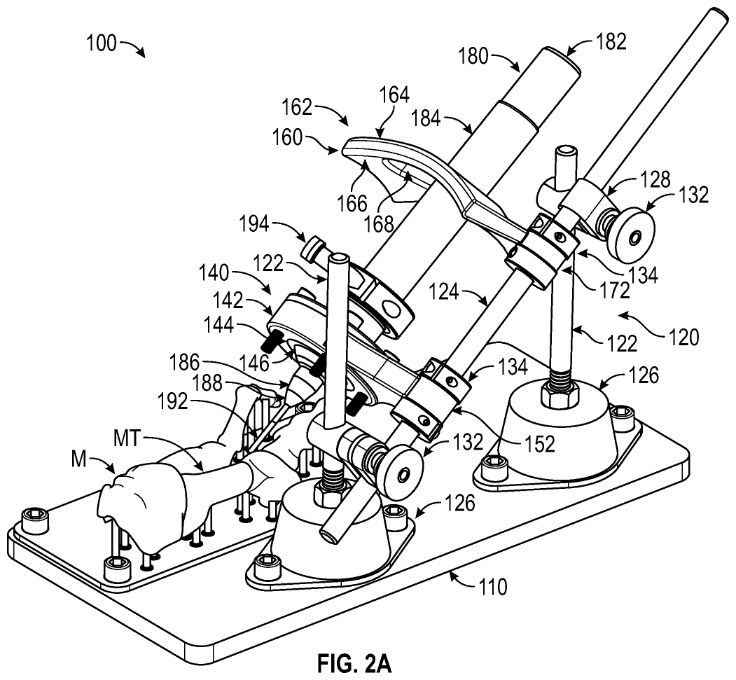

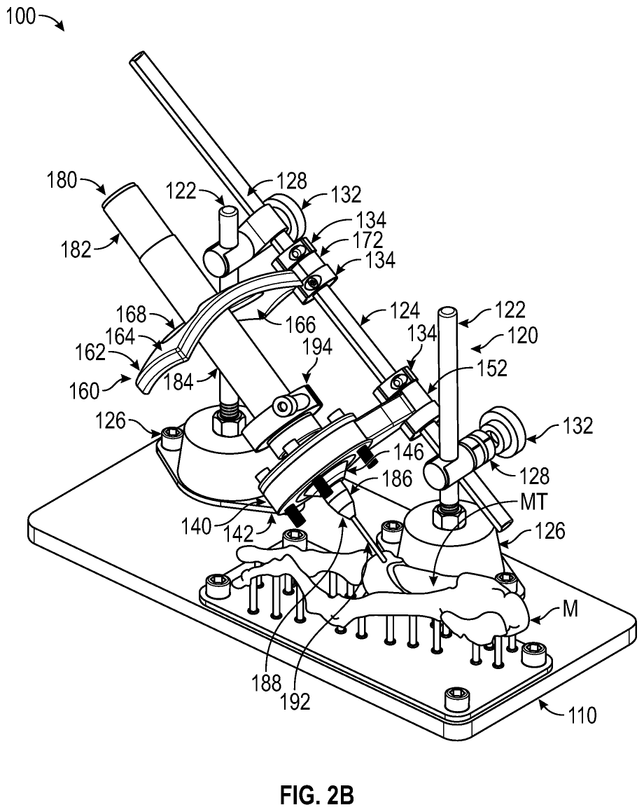

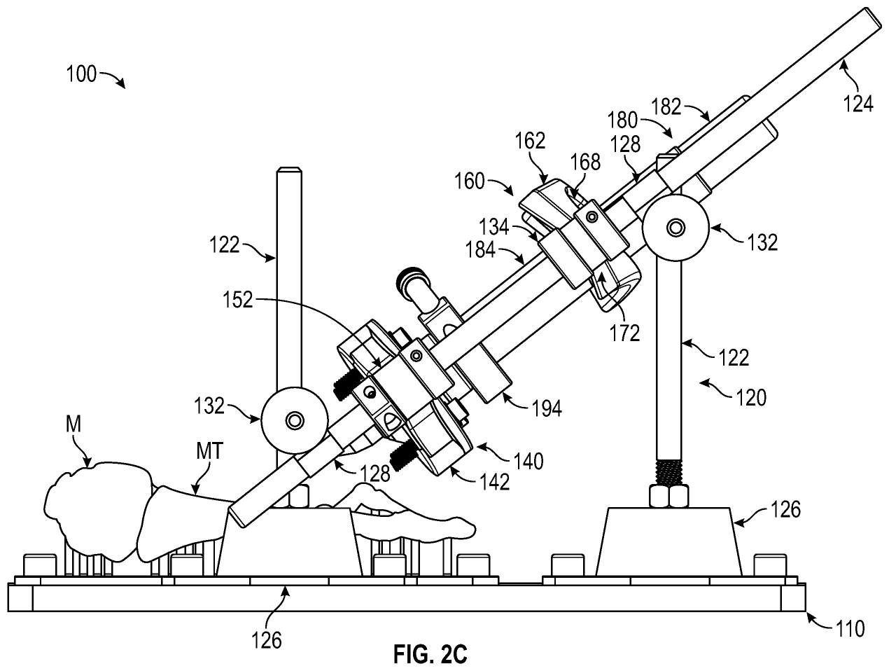

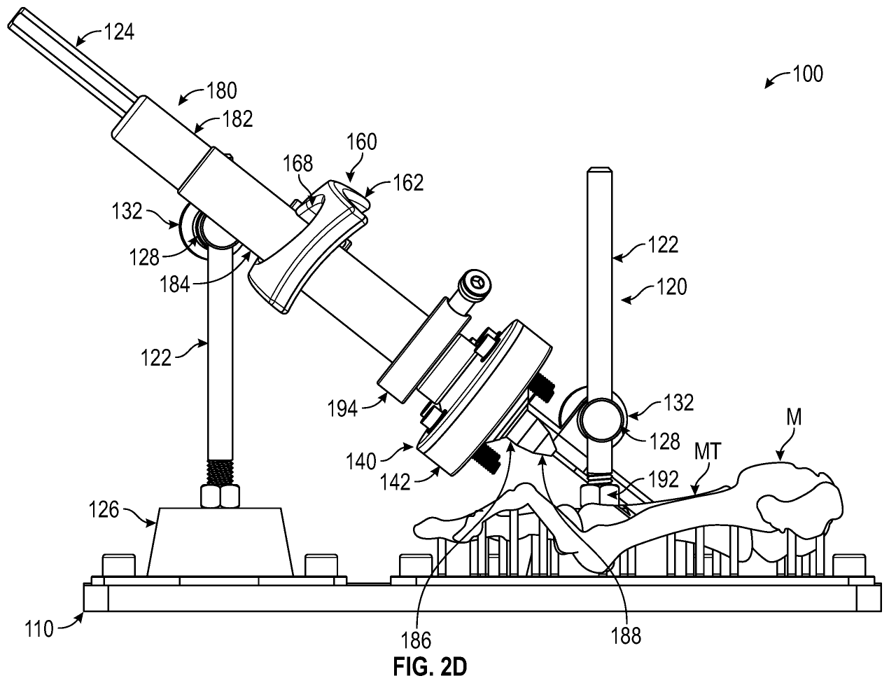

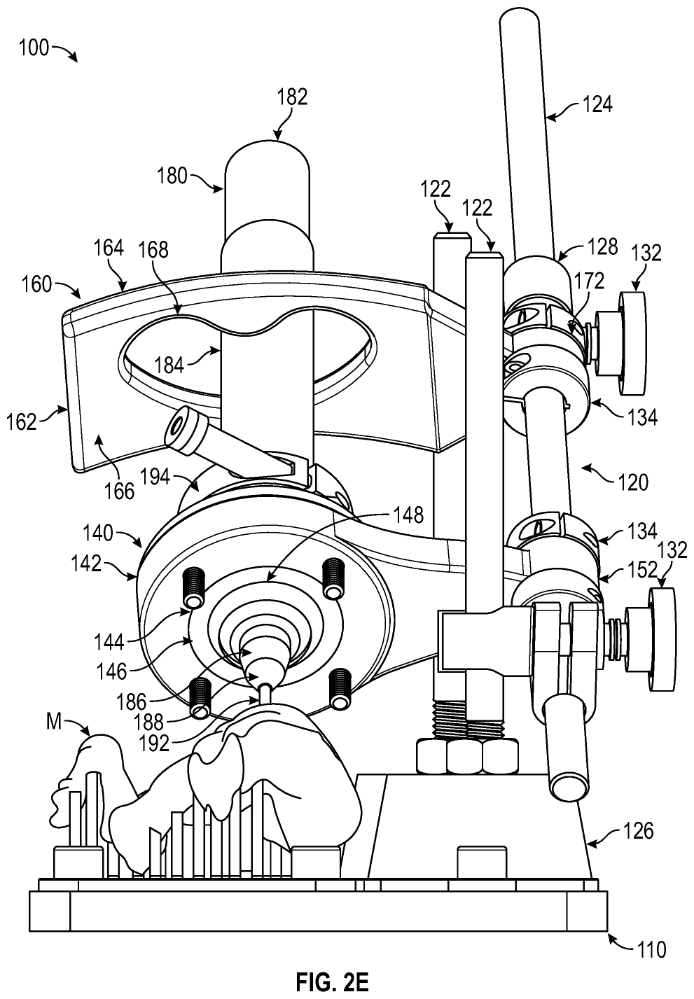

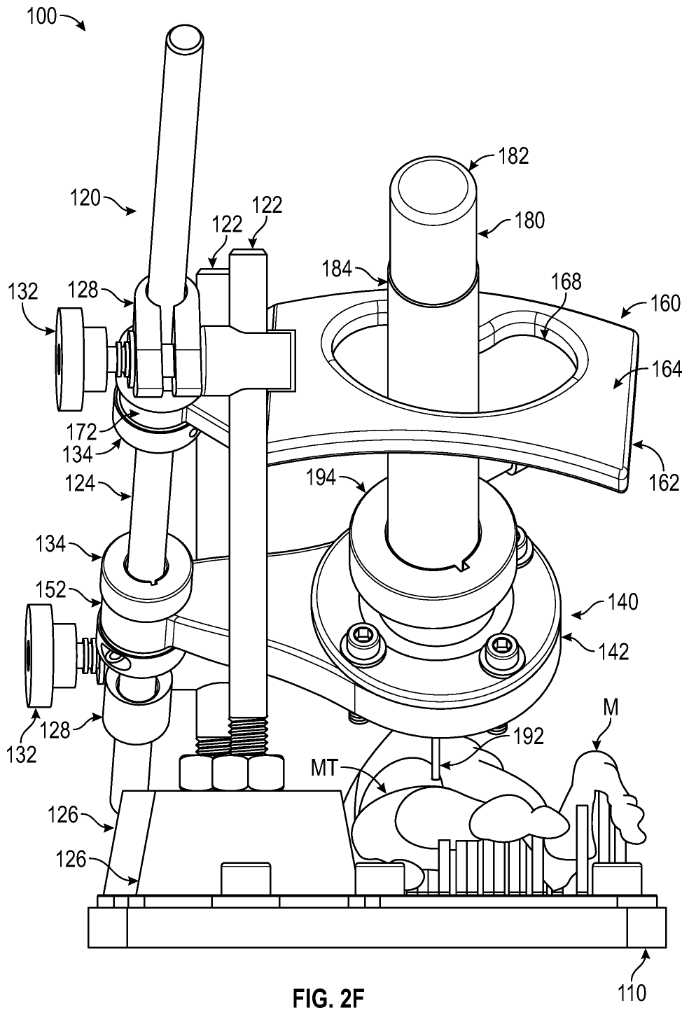

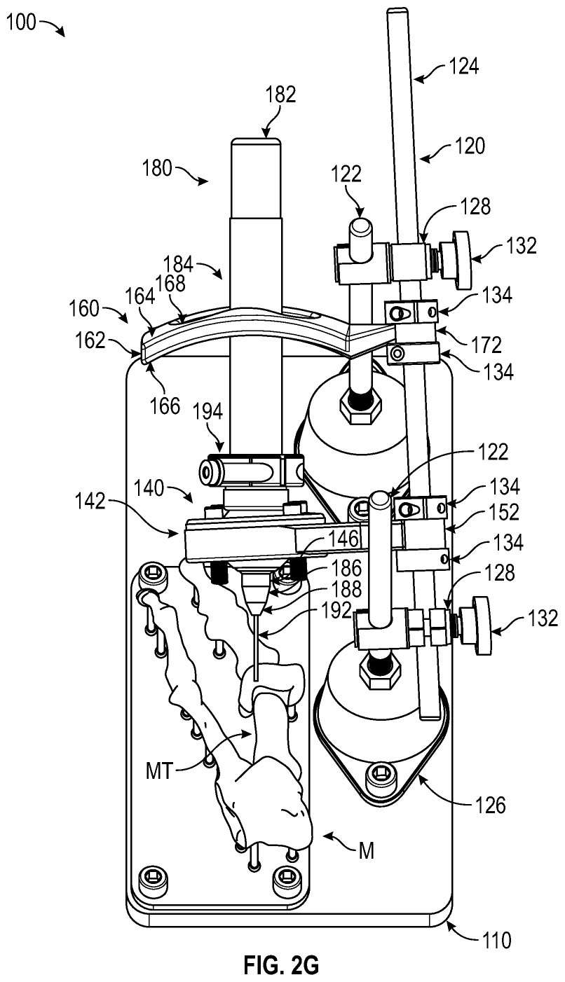

FIGS. 2A-2G illustrate an example jig 100 (which also may be referred to as a “surgical cutting jig” or an “osteotomy jig”) and components thereof in accordance with implementations of the disclosure. As shown, the jig 100 may be used for guiding a cutting instrument 180 for controlled surgical cutting of a body portion of a patient. As described herein, the jig 100 may be adapted to provide controlled surgical cutting of various body portions of a patient in accordance with different types of surgical procedures. According to the illustrated example, the jig 100 and the cutting instrument 180 are shown positioned relative to a model M of a portion of a patient's foot for purposes of performing an osteotomy on the metatarsal MT for bunion correction. It will be appreciated that this is merely one example use of the jig 100. In some instances, the jig 100 and the cutting instrument 180 may be considered to collectively form a system for controlled surgical cutting of a body portion.

As shown, the cutting instrument 180 may include a driver 182 and a cutting burr 192 coupled to the driver 182. The driver 182 may be formed as an elongate assembly having a proximal portion 184, a distal portion 186, and a collet 188. As described below, the proximal portion 184 and the distal portion 186 may be configured for interacting with respective portions of the jig 100, when the cutting instrument 180 is mounted to the jig 100, which control movement of the proximal portion 184 and the distal portion 186, respectively, to provide controlled, precise cutting using the cutting burr 192. The collet 188 may be configured for removably securing the cutting burr 192, as well as other types of cutting tips, to the driver 182. It will be appreciated that the depicted configuration of the cutting instrument 180 is merely one example, and that various other types of cutting instruments may be used in other implementations.

As shown, the jig 100 may include a base 110, a support structure 120, an actuation assembly 140 (also referred to as an angulation assembly), and a guide 160. The base 110 may be configured for supporting and positioning the other components of the jig 100 for use. As shown, the base 110 may be formed as a baseplate having a planar shape, although other configurations of the base 110 may be used. In some implementations, the base 110 may be configured for attaching to a bed, a table, or other type of structure configured for supporting the patient thereon.

The support structure 120 may be configured for positioning relative to the patient and for supporting the actuation assembly 140 and the guide 160. In some implementations, as shown, the support structure 120 may be adjustable, allowing for the position and orientation of the actuation assembly 140 and the guide 160 to be adjusted, for example, to accommodate the anatomy of the patient and the surgical procedure to be performed. As shown, the support structure 120 may be attached to the base 110 and supported thereby. In some implementations, the base 110 may be omitted, and the support structure 120 may be configured for attaching to a bed, a table, or other type of structure configured for supporting the patient thereon.

As shown, the support structure 120 may include a pair of first supports 122 (which also may be referred to as “vertical supports”), a second support 124 (which also may be referred to as a “transverse support”), a pair of mounts 126, a pair of coupling assemblies 128, a pair of locking assemblies 132, and a plurality of clamps 134. The first supports 122 may extend parallel to one another and may be fixedly coupled to the base 110 via the mounts 126. The second support 124 may be coupled to each of the first supports 122 and extend transverse to each of the first supports 122. Each of the coupling assemblies 128 may adjustably couple the second support 124 to one of the first supports 122. As shown, each of the coupling assemblies 128 may allow adjustment of the second support 124 and the respective first support 122 relative to one another, by translation and/or rotation. Each of the locking assemblies 132 may be configured for moving between a locked configuration, in which the locking assembly 132 prevents adjustment of the second support 124 and the respective first support 122 relative to one another, and an unlocked configuration, in which the locking assembly 132 permits adjustment of the second support 124 and the respective first support relative to one another via the respective coupling assembly 128. Each of the clamps 134 may be configured for moving between a locked configuration and an unlocked configuration for preventing and permitting movement of the angulation assembly 140 or the guide 160 relative to the support structure 120, as described below.

The angulation assembly 140 may be coupled to the support structure 120. In some implementations, the angulation assembly 140 may be fixedly coupled to the support structure 120. In other implementations, as shown, the angulation assembly 140 may be movably coupled to the support structure 120, permitting movement of the angulation assembly 140 relative to the support structure 120 when the respective clamps 134 are in the unlocked configuration. When the respective clamps 134 are in the locked configuration, they may prevent movement of the angulation assembly 140 relative to the support structure 120. As shown, the angulation assembly 140 may be configured for receiving the distal portion 186 of the driver 182 therein and controlling angular movement of the cutting instrument 180 about a central point defined by the angulation assembly 140 in a plurality of planes orthogonal to one another.

As shown, the angulation assembly 140 may include a housing 142, a bearing 146, and a mounting ring 152. The housing 142 may define a thru hole 144 extending therethrough. The bearing 146 may define a bore 148 extending therethrough. As shown, the bearing 146 may be positioned at least partially within the housing 142 and centered on the thru hole 144 thereof. In this manner, the distal portion 186 of the driver 182 may extend through the bore 148 and the thru hole 144. In some implementations, as shown, the bearing 146 may be a spherical bearing including an inner ring having a partial-spherical outer surface and an outer ring having a partial-spherical inner surface. In this manner, when the distal portion 186 of the driver 182 is received within the bore 148, the bearing 146 may allow and control angular movement of the cutting instrument 180 about a central point defined by the bearing 146, with the limits of such angular movement being defined by the limits of the spherical bearing. The mounting ring 152 may be coupled to the support structure 120. In some implementations, as shown, the mounting ring 152 may encircle the second support 124 and be configured for translating along the second support 124 when the clamps 134 positioned adjacent the mounting ring 152 are in the unlocked configuration.

The guide 160 may be coupled to the support structure 120. In some implementations, the guide 160 may be fixedly coupled to the support structure 120. In other implementations, as shown, the guide 160 may be movably coupled to the support structure 120, permitting movement of the guide 160 relative to the support structure 120 when the respective clamps 134 are in the unlocked configuration. When the respective clamps 134 are in the locked configuration, they may prevent movement of the guide 160 relative to the support structure 120. As shown, the guide 160 may be configured for receiving the proximal portion 184 of the driver 182 therein and controlling movement of the proximal portion 184 of the driver 182 relative to the guide 160.

As shown, the guide 160 may include a guide plate 162 defining an opening 168 extending therethrough, and a mounting ring 172 coupled to the guide plate 162. The opening 168 may be configured for receiving the proximal portion 184 of the driver 182 therein and controlling movement of the proximal portion 184 of the driver 182 along a first predefined path such that the cutting burr 192 moves along a second predefined path. As shown, the guide plate 162 may have a first surface 164 and a second surface 166 disposed opposite one another, with the opening 168 extending from the first surface 164 to the second surface 166. The first surface 164 may face away from the angulation assembly 140, and the second surface 166 may face toward the angulation assembly 140. In some implementations, the guide plate 162 may have a curved shape such that each of the first surface 164 and the second surface 166 is a curved surface. For example, as shown, the first surface 164 may be a convex surface, and the second surface 166 may be a concave surface. The mounting ring 172 may be coupled to the support structure 120. In some implementations, as shown, the mounting ring 172 may encircle the second support 124 and be configured for translating along the second support 124 when the clamps 134 positioned adjacent the mounting ring 172 are in the unlocked configuration.

Various shapes of the opening 168 may be used to define the first predefined path along which the proximal portion 184 of the driver 182 may be moved and thus also the second predefined path along which the cutting burr 192 moves as a result of the movement of the proximal portion 184 of the driver 182. In some implementations, the opening 168 may have an elongate shape such that each of the first predefined path and the second predefined path has an elongate shape. In some implementations, the opening 168 may have a linear shape such that each of the first predefined path and the second predefined path has a linear shape. In some implementations, the opening 168 may have a contoured shape such that each of the first predefined path and the second predefined path has a contoured shape. In some implementations, the opening 168 may have a curved shape such that each of the first predefined path and the second predefined path has a curved shape. In some implementations, as shown, the opening 168 may have a crescentic shape such that each of the first predefined path and the second predefined path has a crescentic shape. In some implementations, the first predefined path and the second predefined path may have the same shape. In some implementations, the first predefined path may have a first size, and the second predefined path may have a second size that is different than the first size. In some implementations, the second size may be less than the first size. In some implementations, the second predefined path may be inverted relative to the first predefined path. Ultimately, the shape of the opening 168 may be selected such that the cutting burr 192 forms a desired shape of the cut made in the body portion, which may vary based on the particular surgical procedure being performed. In this manner, the guide 160 and the overall jig 100 may have various configurations for different surgical procedures.

In some implementations, the guide 160 and the opening 168 thereof may be configured for a particular procedure. In this manner, a particular configuration of the guide 160 may be used for different patients undergoing the same procedure. In some implementations, the guide 160 and the opening 168 thereof may be configured for a particular procedure and a particular patient. In other words, the guide 160 may be custom manufactured or modified to be patient-specific. In this manner, a particular configuration of the guide 160 may be used for a particular patient undergoing a particular procedure.

An example method of using the jig 100 for guiding the cutting instrument 180 for controlled cutting of a body portion of a patient may first include mounting the cutting instrument 180 to the jig 100. As discussed above, the distal portion 186 of the driver 182 may be received within the bearing 146 of the angulation assembly 140, while the proximal portion 184 of the driver 182 may be received within the opening 168 of the guide 160. In some implementations, the support structure 120 may be adjusted to provide a desired angle of approach of the cutting instrument 180 and the cutting burr 192 thereof toward the body portion of the patient. In some implementations, the support structure 120 may be adjusted to provide a desired cutting depth of the cutting burr 192 into the body portion of the patient. In some implementations, the angulation assembly 140 and/or the guide 160 may be adjusted relative to the second support 124, as discussed above, to provide the desired angle of approach and/or the desired cutting depth. After mounting the cutting instrument 180 to the jig 100, the body portion may be penetrated with the cutting burr 192. Then, the proximal portion 184 of the driver 182 may be moved within the opening 168 of the guide plate 162 along the first predetermined path such that the cutting burr 192 cuts the body portion along the second predefined path. In some implementations, the body portion may be a bone of the patient. For example, the body portion may be a bone of the patient's toes, feet, legs, knees, hips, shoulders, jaw, or spine. In some implementations, the surgical cutting of the body portion may be an osteotomy. It will be appreciated, however, that various configurations of the jig 100 may be used for guiding the cutting instrument 180 for controlled cutting of various body portions of a patient, according to different types of surgical procedures.

In some implementations, the jig 100 may be used for guiding the cutting instrument 180 for performing metatarsal osteotomies of a patient's foot, such as distal first metatarsal osteotomies related to bunion surgery, midshaft first metatarsal osteotomies related to bunion surgery, or proximal first metatarsal osteotomies related to bunion surgery. In some implementations, the jig 100 may be used for guiding the cutting instrument 180 for performing lesser metatarsal osteotomies, such as distal short oblique metatarsal osteotomies related to hammertoe surgery and metatarsalgia, midshaft central metatarsal osteotomies related to foot deformity, including metatarsus adductus, or proximal central metatarsal osteotomies related to midfoot deformity. In some implementations, the jig 100 may be used for guiding the cutting instrument 180 for performing fifth metatarsal osteotomies, such as distal fifth metatarsal osteotomies related to tailor's bunion, central fifth metatarsal osteotomies related to tailor's bunion, or proximal fifth metatarsal osteotomies related to tailor's bunion. In some implementations, the jig 100 may be used for guiding the cutting instrument 180 for performing calcaneal osteotomies, such as calcaneal realignment surgery, calcaneal realignment surgery as related to valgus malalignment of the heel, or calcaneal realignment surgery as related to varus malalignment of the heel. In some implementations, the jig 100 may be used for guiding the cutting instrument 180 for performing phalangeal osteotomies, such as first proximal and distal phalangeal osteotomies of the hallux. In some implementations, the jig 100 may be used for guiding the cutting instrument 180 for performing wedge resection and alignment procedures for fusion of the foot and ankle, such as first metatarsal phalangeal joint fusion, tarsal metatarsal joint fusion, or midfoot and rearfoot realignment joint fusion.

Although specific implementations of the disclosure have been described, one of ordinary skill in the art will recognize that numerous other modifications and alternative implementations are within the scope of the disclosure. For example, any of the functionality and/or processing capabilities described with respect to a particular device or component may be performed by any other device or component. Further, while various illustrative implementations and architectures have been described in accordance with implementations of the disclosure, one of ordinary skill in the art will appreciate that numerous other modifications to the illustrative implementations and architectures described herein are also within the scope of this disclosure.

Although implementations have been described in language specific to structural features and/or methodological acts, it is to be understood that the disclosure is not necessarily limited to the specific features or acts described. Rather, the specific features and acts are disclosed as illustrative forms of implementing the implementations. Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or steps. Thus, such conditional language is not generally intended to imply that features, elements, and/or steps are in any way required for one or more implementations or that one or more implementations necessarily include logic for deciding, with or without user input or prompting, whether these features, elements, and/or steps are included or are to be performed in any particular implementation. The term “based at least in part on” and “based on” are synonymous terms which may be used interchangeably herein.

What is claimed is:

1. A jig for guiding a cutting instrument for controlled surgical cutting of a body portion of a patient, the cutting instrument comprising a driver and a cutting burr coupled to the driver, the jig comprising:
an adjustable support structure configured for positioning relative to the patient;
an angulation assembly coupled to the adjustable support structure, the angulation assembly configured for receiving a distal portion of the driver therein and controlling angular movement of the cutting instrument about a central point defined by the angulation assembly in a plurality of planes orthogonal to one another; and
a guide coupled to the adjustable support structure and spaced apart from the angulation assembly, the guide defining an opening extending therethrough, and the opening configured for receiving a proximal portion of the driver therein and controlling movement of the proximal portion of the driver along a first predefined path such that the cutting burr moves along a second predefined path.

2. The jig of claim 1, wherein the adjustable support structure comprises:
a plurality of supports; and
one or more coupling assemblies each adjustably coupling two of the supports to one another.

3. The jig of claim 2, wherein the plurality of supports comprises:
a pair of first supports extending parallel to one another, and
a second support coupled to each of the first supports and extending transverse to each of the first supports.

4. The jig of claim 3, wherein the first supports each comprise a plurality of first markings, and wherein the second support comprises a plurality of second markings.

5. The jig of claim 4, wherein the first markings correspond to an angle of approach of the cutting instrument toward the body portion of the patient or a cutting depth of the cutting instrument into the body portion of the patient, and wherein the second markings correspond to the angle of approach of the cutting instrument toward the body portion of the patient or the cutting depth of the cutting instrument into the body portion of the patient.

6. The jig of any one of claims 3-5, wherein the one or more coupling assemblies comprises:
a first coupling assembly adjustably coupling the second support to one of the first supports; and
a second coupling assembly adjustably coupling the second support to the other of the first supports.

7. The jig of claim 6, wherein the first coupling assembly is configured for allowing the second support to translate relative to the one of the first supports, and wherein the second coupling assembly is configured for allowing the second support to translate relative to the other of the first supports.

8. The jig of claim 6 or claim 7, wherein the first coupling assembly is configured for allowing the second support to rotate relative to the one of the first supports, and wherein the second coupling assembly is configured for allowing the second support to rotate relative to the other of the first supports.

9. The jig of any one of claims 6-8, wherein the adjustable support structure further comprises:
a first locking assembly configured for moving between a locked configuration, in which the first locking assembly prevents adjustment of the second support and the one of the first supports relative to one another, and an unlocked configuration, in which the first locking assembly permits adjustment of the second support and the one of the first supports relative to one another via the first coupling assembly; and
a second locking assembly configured for moving between a locked configuration, in which the second locking assembly prevents adjustment of the second support and the other of the first supports relative to one another, and an unlocked configuration, in which the second locking assembly permits adjustment of the second support and the other of the first supports relative to one another via the second coupling assembly.

10. The jig of any one of claims 1-9, wherein the adjustable support structure is configured for removably attaching to a bed configured for receiving the patient thereon.

11. The jig of any one of claims 1-9, wherein the adjustable support structure is configured for removably attaching to a table configured for receiving the patient thereon.

12. The jig of any one of claims 1-9, wherein the adjustable support structure is configured for removably attaching to the patient.

13. The jig of any one of claims 1-9, further comprising a base, wherein the adjustable support structure is fixedly attached to the base.

14. The jig of claim 13, wherein the base comprises a baseplate having a planar shape, and wherein the adjustable support structure is fixedly attached to the baseplate by one or more mounts.

15. The jig of any one of claims 1-14, wherein the adjustable support structure is configured for adjusting an angle of approach of the cutting instrument toward the body portion of the patient.

16. The jig of any one of claims 1-15, wherein the adjustable support structure is configured for adjusting a cutting depth of the cutting instrument into the body portion of the patient.

17. The jig of any one of claims 1-16, wherein the angulation assembly is fixedly coupled to the adjustable support structure.

18. The jig of any one of claims 1-16, wherein the angulation assembly is movably coupled to the adjustable support structure.

19. The jig of claim 18, wherein the angulation assembly is movably coupled to a support of the adjustable support structure.

20. The jig of claim 19, wherein the angulation assembly is configured for translating relative to the support of the adjustable support structure.

21. The jig of any one of claims 18-20, wherein the adjustable support structure further comprises one or more clamps configured for moving between a locked configuration, in which the one or more clamps prevents movement of the angulation assembly relative to the adjustable support structure, and an unlocked configuration, in which the one or more clamps permits movement of the angulation assembly relative to the adjustable support structure.

22. The jig of any one of claims 1-21, wherein the angulation assembly comprises a bearing configured for receiving the distal portion of the driver therein and controlling angular movement of the cutting instrument about the central point in the plurality of planes orthogonal to one another.

23. The jig of claim 22, wherein the central point is defined by the bearing.

24. The jig of claim 22 or claim 23, wherein the bearing is a spherical bearing comprising an inner ring having a partial-spherical outer surface, and an outer ring having a partial-spherical inner surface.

25. The jig of any one of claims 22-24, wherein the angulation assembly further comprises a housing, and wherein the bearing is disposed at least partially within the housing.

26. The jig of any one of claims 22-25, wherein the angulation assembly further comprises a mounting ring coupled to the adjustable support structure.

27. The jig of claim 26, wherein the mounting ring encircles a support of the adjustable support structure.

28. The jig of claim 26 or claim 27, wherein the mounting ring is configured for translating along the support of the adjustable support structure.

29. The jig of any one of claims 1-28, wherein the guide is fixedly coupled to the adjustable support structure.

30. The jig of any one of claims 1-28, wherein the guide is movably coupled to the adjustable support structure.

31. The jig of claim 30, wherein the guide is movably coupled to a support of the adjustable support structure.

32. The jig of claim 31, wherein the guide is configured for translating relative to the support of the adjustable support structure.

33. The jig of any one of claims 30-32, wherein the adjustable support structure further comprises one or more clamps configured for moving between a locked configuration, in which the one or more clamps prevents movement of the guide relative to the adjustable support structure, and an unlocked configuration, in which the one or more clamps permits movement of the guide relative to the adjustable support structure.

34. The jig of any one of claims 1-33, wherein the guide comprises a guide plate, and wherein the guide plate defines the opening.

35. The jig of claim 34, wherein the guide plate comprises a first surface and a second surface disposed opposite one another, and wherein the opening extends from the first surface to the second surface.

36. The jig of claim 35, wherein the first surface faces away from the angulation assembly, and wherein the second surface faces toward the angulation assembly.

37. The jig of claim 35 or claim 36, wherein the guide plate has a curved shape such that each of the first surface and the second surface is a curved surface.

38. The jig of any one of claims 35-37, wherein the first surface is a convex surface, and wherein the second surface is a concave surface.

39. The jig of any one of claims 34-38, wherein the guide further comprises a mounting ring coupled to the adjustable support structure.

40. The jig of claim 39, wherein the mounting ring encircles a support of the adjustable support structure.

41. The jig of claim 39 or claim 40, wherein the mounting ring is configured for translating along the support of the adjustable support structure.

42. The jig of any one of claims 1-41, wherein the opening has an elongate shape such that each of the first predefined path and the second predefined path has an elongate shape.

43. The jig of any one of claims 1-41, wherein the opening has a linear shape such that each of the first predefined path and the second predefined path has a linear shape.

44. The jig of any one of claims 1-41, wherein the opening has a contoured shape such that each of the first predefined path and the second predefined path has a contoured shape.

45. The jig of any one of claims 1-41, wherein the opening has a curved shape such that each of the first predefined path and the second predefined path has a curved shape.

46. The jig of any one of claims 1-41, wherein the opening has a crescentic shape such that each of the first predefined path and the second predefined path has a crescentic shape.

47. The jig of any one of claims 1-41, wherein the first predefined path and the second predefined path have the same shape.

48. The jig of claim 47, wherein the first predefined path has a first size, and wherein the second predefined path has a second size that is different than the first size.

49. The jig of claim 48, wherein the second size is less than the first size.

50. The jig of any one of claims 47-49, wherein the second predefined path is inverted relative to the first predefined path.

51. A system for controlled surgical cutting of a body portion of a patient, the system comprising:
a cutting instrument comprising:
a driver; and
a cutting burr coupled to the driver; and
a jig configured for mounting the cutting instrument thereto, the jig comprising:
an adjustable support structure configured for positioning relative to the patient;
an angulation assembly coupled to the adjustable support structure, the angulation assembly configured for receiving a distal portion of the driver therein and controlling angular movement of the cutting instrument about a central point defined by the angulation assembly in a plurality of planes orthogonal to one another; and
a guide coupled to the adjustable support structure and spaced apart from the angulation assembly, the guide defining an opening extending therethrough, and the opening configured for receiving a proximal portion of the driver therein and controlling movement of the proximal portion of the driver along a first predefined path such that the cutting burr moves along a second predefined path.

52. The system of claim 51, wherein the adjustable support structure comprises:
a plurality of supports;
one or more coupling assemblies each adjustably coupling two of the supports to one another; and
one of more locking assemblies each configured for moving between a locked configuration, in which the locking assembly prevents adjustment of two of the supports relative to one another, and an unlocked configuration, in which the locking assembly permits adjustment of the two of the supports relative to one another via the one or more coupling assemblies.

53. The system of claim 51 or claim 52, wherein the angulation assembly comprises a bearing configured for receiving the distal portion of the driver therein and controlling angular movement of the cutting instrument about the central point in the plurality of planes orthogonal to one another.

54. The system of claim 53, wherein the bearing is a spherical bearing comprising an inner ring having a partial-spherical outer surface, and an outer ring having a partial-spherical inner surface.

55. The system of any one of claims 51-54, wherein the opening has an elongate shape such that each of the first predefined path and the second predefined path has an elongate shape.

56. The system of any one of claims 51-54, wherein the opening has a linear shape such that each of the first predefined path and the second predefined path has a linear shape.

57. The system of any one of claims 51-54, wherein the opening has a contoured shape such that each of the first predefined path and the second predefined path has a contoured shape.

58. The system of any one of claims 51-54, wherein the opening has a curved shape such that each of the first predefined path and the second predefined path has a curved shape.

59. The system of any one of claims 51-54, wherein the opening has a crescentic shape such that each of the first predefined path and the second predefined path has a crescentic shape.

60. The system of any one of claims 51-54, wherein the first predefined path and the second predefined path have the same shape, and wherein the second predefined path is inverted relative to the first predefined path.

61. A method for guiding a cutting instrument for controlled surgical cutting of a body portion of a patient, the cutting instrument comprising a driver and a cutting burr coupled to the driver, the method comprising:
mounting the cutting instrument to a jig, the jig comprising:
an adjustable support structure configured for positioning relative to the patient;
an angulation assembly coupled to the adjustable support structure, the angulation assembly receiving a distal portion of the driver therein and controlling angular movement of the cutting instrument about a central point defined by the angulation assembly in a plurality of planes orthogonal to one another; and
a guide coupled to the adjustable support structure and spaced apart from the angulation assembly, the guide defining an opening extending therethrough, and the opening receiving a proximal portion of the driver therein and controlling movement of the proximal portion of the driver along a first predefined path such that the cutting burr moves along a second predefined path;
penetrating the body portion of the patient with the cutting burr; and
moving the proximal portion of the driver within the opening along the first predefined path such that the cutting burr cuts the body portion along the second predefined path.

62. The method of claim 61, wherein the body portion of the patient is a bone of the patient.

63. The method of claim 61, wherein the body portion is a bone of the patient's toes, feet, legs, knees, hips, shoulders, jaw, or spine.

64. The method of claim 61, wherein the surgical cutting is an osteotomy.

65. The method of any one of claims 61-64, wherein the opening has an elongate shape such that each of the first predefined path and the second predefined path has an elongate shape.

66. The method of any one of claims 61-64, wherein the opening has a linear shape such that each of the first predefined path and the second predefined path has a linear shape.

67. The method of any one of claims 61-64, wherein the opening has a contoured shape such that each of the first predefined path and the second predefined path has a contoured shape.

68. The method of any one of claims 61-64, wherein the opening has a curved shape such that each of the first predefined path and the second predefined path has a curved shape.

69. The method of any one of claims 61-64, wherein the opening has a crescentic shape such that each of the first predefined path and the second predefined path has a crescentic shape.

70. The method of any one of claims 61-64, wherein the first predefined path and the second predefined path have the same shape, and wherein the second predefined path is inverted relative to the first predefined path.

71. The method of claim 61, wherein the surgical cutting comprises performing a metatarsal osteotomy of the patient's foot.

72. The method of claim 61, wherein the surgical cutting comprises performing a lesser metatarsal osteotomy of the patient's foot.

73. The method of claim 61, wherein the surgical cutting comprises performing a fifth metatarsal osteotomy of the patient's foot.

74. The method of claim 61, wherein the surgical cutting comprises performing a calcaneal osteotomy of the patient's foot.

75. The method of claim 61, wherein the surgical cutting comprises performing a phalangeal osteotomy of the patient's foot.

76. The method of claim 61, wherein the surgical cutting comprises performing a wedge resection and alignment procedure for fusion of the patient's foot and ankle.
